(19) United States
(12) Patent Application Publication
(10) Pub. No.: US 2025/0288337 A1
(43) Pub. Date: Sep. 18, 2025

(54) VERTEBRAL CEMENT INJECTION SYSTEM AND METHOD OF USE

(71) Applicants: Mark Frenkel, Naples, FL (US); Mark Gerber, Naples, FL (US)

(72) Inventors: Mark Frenkel, Naples, FL (US); Mark Gerber, Naples, FL (US)

(21) Appl. No.: 19/080,690

(22) Filed: Mar. 14, 2025

Related U.S. Application Data
(60) Provisional application No. 63/565,655, filed on Mar. 15, 2024.

(51) Int. Cl. A61B 17/88

(52) U.S. Cl. CPC A61B 17/8811

(57) ABSTRACT

A system for injecting a bone reinforcement composition into at least one bone proximate a joint, wherein the bone reinforcement composition is injected to reinforce the region of the bone proximate the joint. The bone reinforcement composition is injected using a bone reinforcement composition delivery system. An aperture through an implant is employed to locate the delivery system in an appropriate location of the respective bone. A secondary alignment tool can be employed to aid in registering the delivery system with the aperture. For a vertebral interbody device, the aperture is provided through a vertebral flange. An interbody insertion tool can include a primary registration feature to align the delivery system with the aperture. Once aligned, a needle of the delivery system is inserted into the bone and a volume of the bone reinforcement composition is injected into the bone.

### CROSS-REFERENCE TO RELATED APPLICATION

The present application claims the priority benefit of U.S. provisional patent application 63/565,655 filed Mar. 15, 2024, the disclosure of which is incorporated herein by reference.

### TECHNICAL FIELD

The present disclosure generally relates to a system of dispensing bone cement into vertebrae during a surgical procedure. More specifically, the system employs an aperture passing through a disc retention flange extending substantially perpendicularly from an intervertebral spacer. A cement dispensing device passes through the aperture and a needle of the cement dispensing device is embedded into the vertebrae. Once the needle is embedded at the desired location, the cement is delivered into the vertebrae via the cement dispensing device. The cement is dispensed to provide a reinforcement to the bone to support the intervertebral spacer.

### BACKGROUND

Degenerative disc disease describes the gradual failure of the disc to perform its function, resulting in worsened range of motion and back pain. Degenerative disc disease can be attributed to aging, overloading of the spine, and possibly genetic factors. The disc is an avascular structure, which makes it susceptible to damage and inability for reliable regeneration. This explains the wide prevalence of degenerative disc diseases.

Physiologically, the disc acts as a shock absorber between adjacent vertebrae. The disc also plays a role in maintaining spinal alignment and facilitating range of motion. Degeneration and collapse of the intervertebral disc cause stress across the facet joint, impingement on neural structures, and strain on paraspinal muscles from loss of alignment. Degenerative disc disease can occur at any point across the spine; however it is most common in the cervical and lumbar regions, causing neck and back pain, respectively. Although disabling, the majority of patients experience gradual resolution of symptoms without need for surgical intervention.

There are two primary procedures for addressing a degenerative disc between two adjacent vertebrae. One procedure involves fusing the adjacent vertebrae. A second procedure involves inserting an artificial disc between the adjacent vertebrae.

In spinal fusion surgery there are several different anatomic approaches to the spine. Some of these include posterior, lateral, or anterior approaches where the surgeon accesses the disc space directly. In interbody fusion procedures, a portion of the disc space is cleared out and an interbody device is inserted in its place. In the lumbar spine specifically, it is commonplace for the surgeon to also place pedicle screws from a posterior approach to increase stability of the construct. In addition, patients with osteopenia or osteoporosis may have an increased risk of vertebral body fracture and graft subsidence and so vertebroplasty is occasionally performed in conjunction with the procedure. This can be done through fenestrated pedicle screws that act as a cannula through which to pass bone cement, or by placing a trocar directly into the vertebral body.

Graft subsidence is a sinking of a body with a higher elasticity modulus, such as a graft, a cage, a spacer, and the like, in a body characterized by a lower elasticity modulus, such as a vertebral body, resulting in three-dimensional changes of the spinal geometry. A magnitude of subsidence is directly proportional to a load pressure and to a difference between the elasticity modules, but inversely proportional to the area of the graft-bed interface. Both biological and mechanical qualities of the graft-bed interface are important for the subsidence process. Any excessive subsidence decreases the interbody space and produces both local and general kyphotization of the spine. This may cause destabilization of the screw-plate and/or screw-bone interfaces, such as pulling out, altered angulation, or breakage of the screws, or any other undesirable destabilizing change.

Although there is no equivalent to the implant endplate in the natural disc, they are a component in every disc implant and play a role in stabilization. Endplates can be made up of alloys from cobalt-chrome, stainless steel, titanium, or a metal and ceramic composite. The osteoconductive nature of titanium enhances the chance of solid adhesion with the adjacent vertebrae. Furthermore, additional features of the endplate aid in stabilization and are in the form of spikes, keels, or screws. Although they support in fixation, these features do have a risk of impacting the vertebral endplate and can impose a challenge for revision.

Artificial discs aim to emulate the qualities of natural discs to allow them to perform the same function. Artificial discs can be classified according to their structure as articulating or non-articulating. Articulating implants are composed of 2 or 3 solid discrete components that are combined in a ball-in-socket or ball-in-trough configuration.

Another approach to reproduce the function of the natural disc is to emulate its structure. The field of tissue engineering aims at creating scaffolds that, once impregnated with a cellular milieu, develop into a viable material that resembles the natural disc tissue at a structural level.

As described above, any of a variety of intervertebral devices is inserted between adjacent vertebrae, replacing natural discs. Numerous factors determine which of the intervertebral devices are used. One of the considerations is the quality of the bone of each of the vertebrae. If the quality or reliability of the bone can be improved, the overall long-term outcome of the procedure is also improved.

### SUMMARY

The present disclosure generally relates to methods of reinforcing bone, anatomical elements, etc. More specifically, procedures can be performed to reinforce vertebrae in a region proximate an intervertebral contacting surface of the vertebrae, a weakened region, a load-bearing region, a region receiving one or more anchors, or other regions.

In some embodiments, the present disclosure relates to a method of injecting cement into a vertebra, the method comprising steps of inserting an implant device to address a defective vertebral disc in an appropriate position respective to the vertebra; aligning a delivery needle of a bone reinforcement composition delivery system to the vertebra using a feature integral with the implant device, the bone reinforcement composition delivery system comprising a delivery needle; and dispensing a volume of a bone reinforcement composition into the vertebra in a location proximate an intervertebral surface of the vertebra.

In one aspect, the feature integral with the implant device is at least one of (a) an aperture formed through a vertebral flange, (b) an aperture formed through a vertebral flange extending outward from an intervertebral device, (c) a bore extending into the intervertebral device, (d) a tool attached to the bore extending into the intervertebral device, or (e) a tool rotationally attached to the bore extending into the intervertebral device.

In some embodiments, the present disclosure relates to a method of injecting cement into a vertebra, the method comprising steps of inserting a vertebral flange against a side of at least one vertebra, the vertebral flange comprising at least one aperture, the aperture located proximate an intervertebral surface of the vertebra; inserting a bone reinforcement composition delivery system through a respective aperture, the bone reinforcement composition delivery system comprising a delivery needle; and dispensing a volume of a bone reinforcement composition into the vertebra in a location proximate the intervertebral surface of the vertebra.

In some embodiments, the bone reinforcement composition is any suitable composition capable of reinforcing material of a bone.

In another embodiment, the bone reinforcement composition is a cement.

In yet another embodiment, the bone reinforcement composition is a bone cement.

In yet another embodiment, the vertebral flange is secured to a vertebrae.

In yet another embodiment, the vertebral flange is secured to an intervertebral device.

In yet another embodiment, the vertebral flange is integral with the intervertebral device.

In yet another embodiment, the vertebral flange is designed to contact one vertebra.

In yet another embodiment, the vertebral flange is designed to contact a first vertebra and a second, adjacent vertebra.

In yet another embodiment, the system includes a first vertebral flange designed to contact a first vertebra and a second vertebral flange designed to contact a second, adjacent vertebra.

In another embodiment, the intervertebral spacer is in a form of an artificial disc.

In yet another embodiment, the intervertebral spacer is in a form of an intervertebral device associated with a fusion process.

In yet another embodiment, the method further comprises a step of employing a cement delivery system guide apparatus.

In yet another embodiment, the method further comprises a step of employing the cement delivery system guide apparatus, wherein the cement delivery system guide apparatus is detachably attached to the vertebral flange.

In yet another embodiment, the method further comprises a step of employing the cement delivery system guide apparatus, wherein the cement delivery system guide apparatus is threadably attached to the vertebral flange.

In yet another embodiment, the method further comprises a step of employing the cement delivery system guide apparatus, wherein the cement delivery system guide apparatus is detachably attached to the intervertebral device.

In yet another embodiment, the method further comprises a step of employing the cement delivery system guide apparatus, wherein the cement delivery system guide apparatus is threadably attached to the intervertebral device.

In yet another embodiment, the method further comprises a step of employing the cement delivery system guide apparatus, wherein the cement delivery system guide apparatus is integral with an installation tool for installing the intervertebral device.

In yet another embodiment, the cement delivery system guide apparatus is designed to rotate when installed.

In yet another embodiment, the cement delivery system guide apparatus is designed to rotate 180 degrees when installed.

In yet another embodiment, the cement delivery system guide apparatus is designed to rotate at least 180 degrees when installed.

In yet another embodiment, the cement delivery system guide apparatus has multiple guide apertures.

In yet another embodiment, the cement delivery system guide apparatus includes at least two cement delivery system guide apertures, wherein one guide aperture is located to align the cement delivery system with a first vertebra and an opposite, second guide aperture is located to align the cement delivery system with a second, adjacent vertebra, thus providing access to each of the first and second vertebrae simultaneously.

In yet another embodiment, the cement delivery system guide apparatus includes at least two cement delivery system guide features, wherein one guide feature is located to align the cement delivery system with a first vertebra and an opposite, second guide aperture is located to align the cement delivery system with a second, adjacent vertebra, thus providing access to each of the first and second vertebrae simultaneously.

In yet another embodiment, the cement delivery system guide apparatus includes at least two cement delivery system guide features, wherein one guide feature is located to align the cement delivery system with a first vertebra and an opposite, second guide aperture is located to align the cement delivery system with a second, adjacent vertebra, thus providing access to each of the first and second vertebrae simultaneously, wherein the cement delivery system guide apparatus includes a control member assembled thereto enabling a sliding motion.

In yet another embodiment, the cement delivery system guide apparatus has a guide tube extending downward to stay in contact with one of the vertebral body or flange aperture to prevent encroachment of soft tissue when inserting and removing working elements.

In yet another embodiment, the vertebral flange system includes a first aperture in a first vertebral flange and a second aperture in a second vertebral flange.

In yet another embodiment, the individual vertebral flange includes multiple cement injection registration apertures.

In yet another embodiment, the individual vertebral flange includes multiple cement injection registration apertures, wherein each cement injection registration aperture provides for a different respective site for injection of cement at several different points in the vertebral body.

In yet another embodiment, the vertebral flange may be a component of a plating system used to secure the intervertebral device to vertebrae.

In yet another embodiment, the vertebral flange may be a component of a plating system used to secure two adjacent vertebrae together.

In yet another embodiment, the vertebral flange retains the intervertebral device in position, the vertebral flange being secured in position by inserting a fastening device through the cement injection registration aperture.

In yet another embodiment, the vertebral flange retains the intervertebral device in position, the vertebral flange being secured in position by inserting a threaded fastening device through the cement injection registration aperture and threadably secured to the vertebra.

In yet another embodiment, the vertebral flange retains the intervertebral device in position, the vertebral flange being secured in position by inserting a fastening device through the cement injection registration aperture and secured to the vertebra.

In yet another embodiment, the vertebral flange further comprises a threading through the aperture.

In yet another embodiment, the vertebral flange further comprises a threading through the aperture, wherein a threaded cannula is threadably secured to the threaded aperture of the vertebral flange.

In yet another embodiment, the vertebral flange may be a component of a plating system used to secure the intervertebral device in position, wherein, when implanted, the vertebral flange is secured by inserting a fastening device through the cement injection registration aperture.

In yet another embodiment, the vertebral flange includes elements to aid in the retraction of tissue from the contacting surface of the vertebral body.

In yet another embodiment, the vertebral flange system is formed comprising a vertebral flange that extends to sufficiently contact sides of two adjacent vertebrae. The vertebral flange includes a first aperture at a location aligned proximate a vertebral contacting surface of the first vertebra and a second aperture at a location aligned proximate a vertebral contacting surface of the second, adjacent vertebra.

In yet another embodiment, the method includes a step of rotating the cement delivery system guide apparatus from the first vertebra in the vertebral flange to a position aligning the cement delivery system guide apparatus with the second aperture in the vertebral flange and the second vertebra.

In yet another embodiment, the method includes steps of removing the cement delivery system from the first vertebra and the first aperture of the vertebral flange; rotating the cement delivery system guide apparatus to align with the second aperture of the vertebral flange; inserting the cement delivery system through the second aperture of the vertebral flange and into the second vertebra; and dispensing cement into the second vertebra in a location proximate the respective intervertebral surface of the second vertebra.

In yet another aspect, the method includes steps of removing the cement delivery system from the first vertebra and the first aperture of the first vertebral flange; rotating the cement delivery system guide apparatus to align with the second aperture of the second vertebral flange; inserting the cement delivery system through the second aperture of the second vertebral flange and into the second vertebra; and dispensing cement into the second vertebra in a location proximate the respective intervertebral surface of the second vertebra.

In yet another aspect, the cement delivery system includes an apparatus to deliver cement through the cement delivery needle.

In yet another aspect, the cement delivery system includes an apparatus to deliver cement through the cement delivery needle, wherein the apparatus is a syringe plunger.

In yet another aspect, the cement delivery system includes an apparatus to deliver cement through the cement delivery needle, wherein the apparatus is a pneumatic drive system.

In yet another aspect, the cement delivery system includes an apparatus to deliver cement through the cement delivery needle, wherein the apparatus is a pump.

In yet another aspect, the cement delivery system includes an apparatus to deliver cement through the cement delivery needle, wherein the apparatus is a displacement pump.

In yet another aspect, the cement delivery system includes an apparatus to deliver cement through the cement delivery needle, wherein the apparatus includes an auger.

In yet another aspect, the cement delivery system includes an apparatus to deliver cement through the cement delivery needle, wherein the apparatus includes an auger, wherein the auger aids in mixing the cement.

In yet another aspect, the cement delivery system includes an apparatus to deliver cement through the cement delivery needle, wherein the cement delivery needle includes a lumen.

In yet another aspect, the cement delivery system includes an apparatus to deliver cement through the cement delivery needle, wherein the cement delivery needle includes a lumen, wherein the lumen is located at an end of the needle.

In yet another aspect, the cement delivery system includes an apparatus to deliver cement through the cement delivery needle, wherein the cement delivery needle includes a lumen, wherein the lumen is located at a beveled end of the needle.

In yet another aspect, the cement delivery system includes an apparatus to deliver cement through the cement delivery needle, wherein the cement delivery needle includes a lumen, wherein the lumen is located through a sidewall of the needle.

In yet another aspect, the cement delivery system includes an apparatus to deliver cement through the cement delivery needle, wherein the cement delivery needle includes a lumen, wherein the lumen is located through a sidewall of the needle at a location proximate a distal end of the needle.

In yet another aspect, the cement delivery system includes an apparatus to deliver cement through the cement delivery needle, wherein the cement delivery needle includes a lumen, wherein the lumen is located through a sidewall of the needle, wherein the sidewall lumen is used to allow for directional delivery of cement.

In yet another aspect, the cement delivery system includes an apparatus to deliver cement through the cement delivery needle, wherein the cement delivery needle may be inserted at varying depths into the vertebrae to allow for delivery of cement to specific regions of the vertebrae.

In yet another aspect, the cement delivery system includes an apparatus to deliver cement through the cement delivery needle, wherein the cement delivery needle includes one or more elements that provide visual feedback for indicating the depth of the needle in the vertebrae.

In yet another aspect, the cement delivery system includes an apparatus to deliver cement through the cement delivery needle, wherein the cement delivery needle includes one or more elements that provide tactile feedback for indicating the depth of the needle in the vertebrae.

In yet another aspect, the cement delivery system includes an apparatus to deliver cement through the cement delivery needle, wherein the cement delivery needle includes one or more elements that provide at least one of tactile feedback and visual feedback for indicating the depth of the needle in the vertebrae.

In yet another aspect, the cement delivery system includes at least one additional apparatus to assist in delivery of the cement, including awls, drills, or balloons.

In yet another aspect, the cement delivery system includes at least one additional apparatus to assist in delivery of the cement, wherein the at least one additional apparatus can include at least one of: an awl, a drill, and a balloon.

In yet another aspect, the cement delivery system includes an apparatus to deliver cement through the cement delivery needle, wherein the cement delivery needle includes a lumen, wherein the lumen is located through a sidewall of the needle at a location proximate a distal end of the needle, wherein the distal end of the needle has a pointed conical shape.

These and other aspects, features, and advantages of one or more disclosed embodiments will become more readily apparent from the attached drawings and the detailed description of the preferred embodiments, which follows.

### BRIEF DESCRIPTION OF THE DRAWINGS

Variants of the technology will hereinafter be described in conjunction with the appended drawings provided to illustrate and not to limit the technology, where like designations denote like elements, and in which:

FIG. 1 is a sectioned front elevation view of a step of removing an intervertebral disc from a space between two exemplary adjacent vertebrae;

FIG. 2 is a sectioned front elevation view of a step of inserting an intervertebral device into the space between the two exemplary adjacent vertebrae;

FIG. 3 is a sectioned front elevation view of the intervertebral device seated into the space between the two exemplary adjacent vertebrae;

FIG. 4 is a side elevation view of the intervertebral device seated into the space between the two exemplary adjacent vertebrae, the illustration introducing optional multiple cement delivery apertures in each flange;

FIG. 5 is a sectioned front elevation view of the intervertebral device seated into the space between the two exemplary adjacent vertebrae, the illustration introducing a cement delivery system and respective guide apparatus;

FIG. 6 is a sectioned front elevation view of the intervertebral device seated into the space between the two exemplary adjacent vertebrae, the illustration introducing the dispensing of cement into each of the two adjacent vertebrae using the cement delivery system and respective guide apparatus;

FIG. 7 is an isometric view of the exemplary vertebral flanges integrated into an exemplary intervertebral device;

FIG. 8 is an exemplary flow diagram of a method of using the cement delivery system;

FIG. 9 is a sectioned front elevation view of a first exemplary intervertebral device installation and cement delivery system guide tool being temporarily assembled to an exemplary intervertebral device, the first exemplary intervertebral device installation and cement delivery system guide tool being used for aligning the cement delivery system with the first vertebra;

FIG. 10 is a sectioned front elevation view of the first exemplary intervertebral device installation and cement delivery system guide tool as introduced in FIG. 9, the first exemplary intervertebral device installation and cement delivery system guide tool being rotated from the first vertebra as illustrated in FIG. 9 and used for aligning the cement delivery system with the second vertebra;

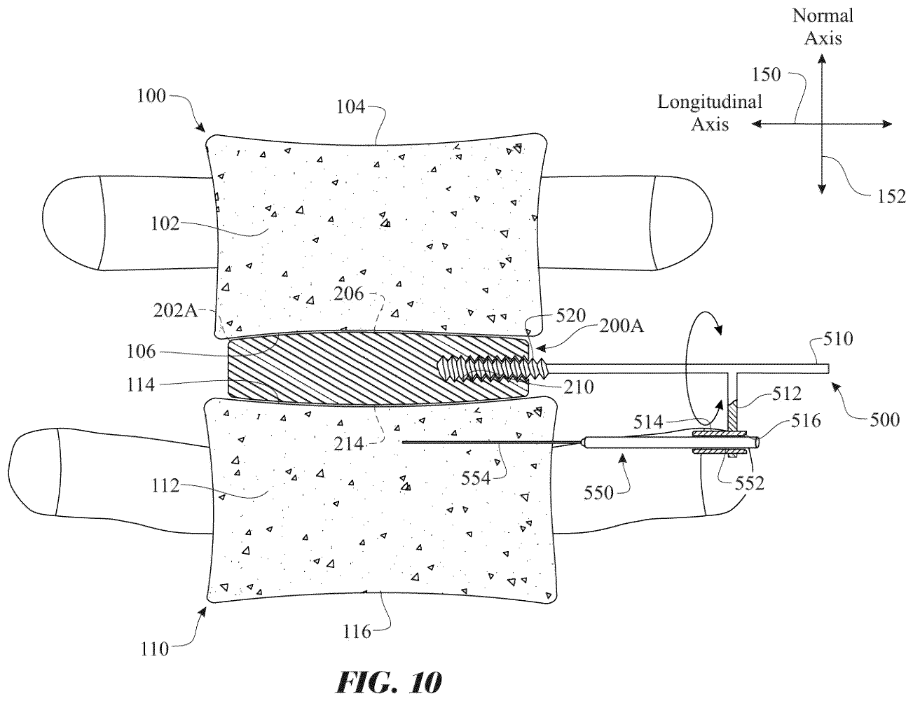

FIG. 11 is a sectioned front elevation view of a second exemplary intervertebral device installation and cement delivery system guide tool being temporarily assembled to an exemplary intervertebral device, the second exemplary intervertebral device installation and cement delivery system guide tool introducing a feature to prevent encroachment of soft tissue when inserting and removing working elements and a depth stop;

FIG. 12 is a sectioned front elevation view of a third exemplary intervertebral device installation and cement delivery system guide tool being temporarily assembled to an exemplary intervertebral device, the third exemplary intervertebral device installation and cement delivery system guide tool introducing a sliding feature for aligning and injecting the cement dispensing needle into the respective vertebrae;

FIG. 13 is a sectioned front elevation view of a modified variant of the third exemplary intervertebral device installation and cement delivery system guide tool originally introduced in FIG. 12, the modified variant introducing a second cement delivery system guide tool, wherein the intervertebral device installation and cement delivery system guide tool simultaneously injects and delivers cement to each of the adjacent vertebrae;

FIG. 14 is a sectioned front elevation view of two different threaded attachment members used for aiding in the delivery of cement to each respective vertebra and for retaining the intervertebral device in position with the two adjacent vertebrae, a first being a solid threaded attachment member and a second being a threaded cannula member;

FIG. 15 is a sectioned front elevation view as originally introduced in FIG. 14, wherein the illustration presents the two respective methods of delivering cement to the vertebra, the first being after removal of a solid threaded attachment member and the second being through the cannula of the threaded cannula member; and

FIG. 16 is a sectioned front elevation view of exemplary vertebral flanges integrated into an exemplary intervertebral device, the exemplary vertebral flanges having a threaded aperture in registration with each respective adjacent vertebra, the threaded aperture being utilized for temporarily retaining a threaded cannula, the threaded cannula providing alignment and registration of the cement delivery system with the respective vertebra.

Like reference numerals refer to like parts throughout the several views of the drawings.

### DETAILED DESCRIPTION

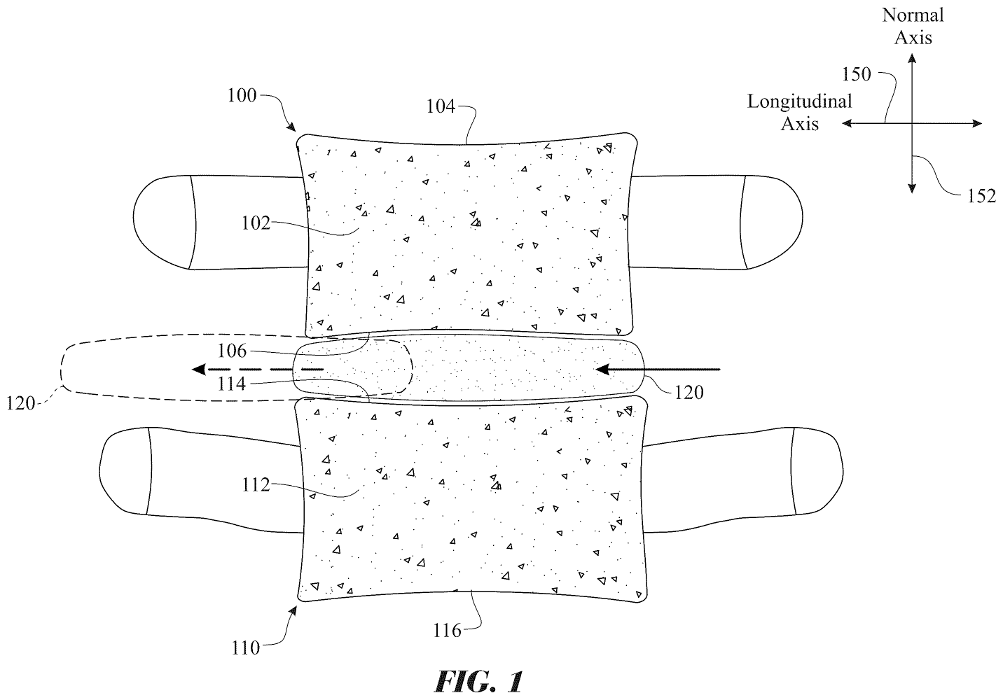

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper,” “lower,” “left,” “rear,” “right,” “front,” “vertical,” “horizontal,” and derivatives thereof shall relate to the embodiment as oriented in FIG. 1. Furthermore, there is no intention to be bound by any expressed or implied theory presented in the preceding technical field, background, brief summary, or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

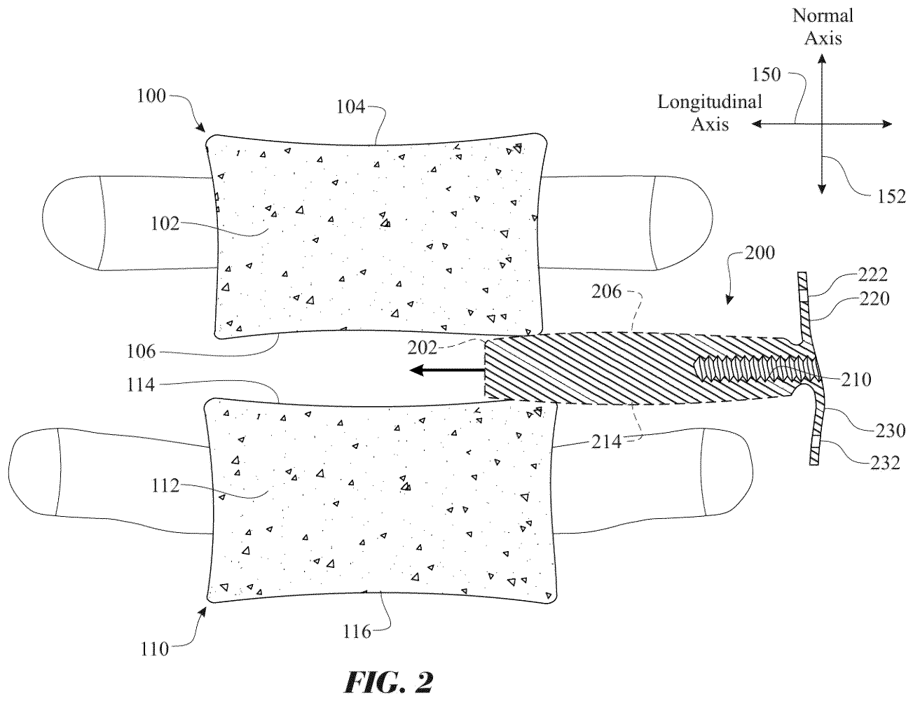

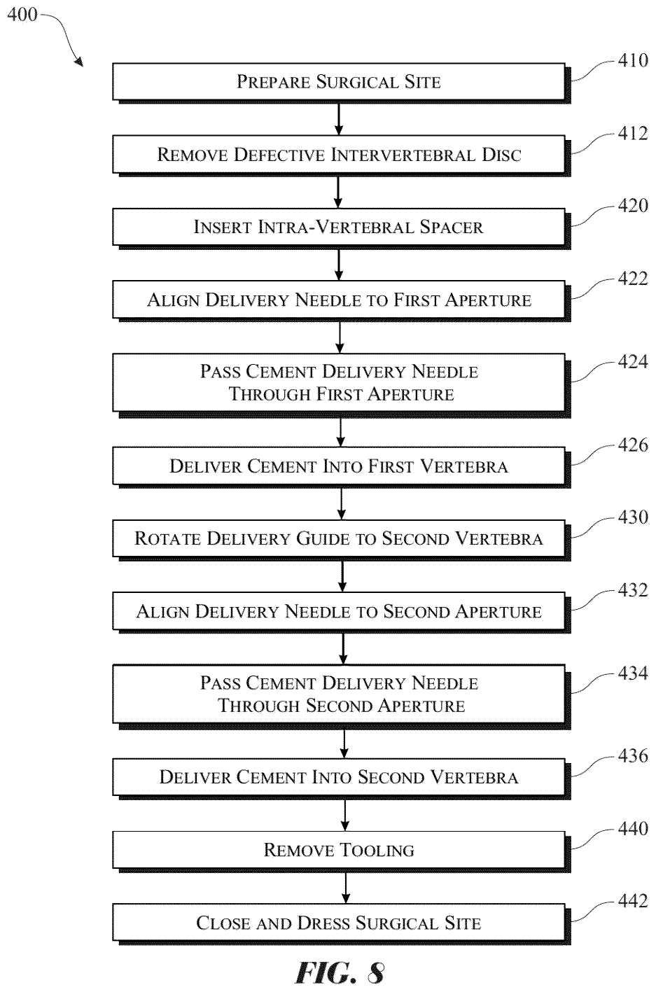

At least some embodiments include a cement delivery system for dispensing bone cement into vertebrae during a procedure to replace a defective intervertebral disc as discussed in connection with FIGS. 1 through 7. A cement delivery system flow diagram 400, detailed in FIG. 8, outlines the process for dispensing bone cement into vertebrae during the procedure to replace the defective intervertebral disc, with supporting drawings being presented in FIGS. 1 through 7.

FIG. 1 is a sectioned front elevation view of an intervertebral joint of a human subject. The intervertebral joint comprises a first joint member 100 and a second joint member 110 having an intervertebral disc 120 provided therebetween. The exemplary first joint member 100 includes a first vertebra 102 having a first joint surface 104 on an upper surface and a second joint surface 106 on a lower surface. The exemplary second joint member 110 includes a second vertebra 112 having a first joint surface 114 on an upper surface and a second joint surface 116 on a lower surface. The second joint surface 106 of the first vertebra 102 and the first joint surface 114 of the second vertebra 112 face one another. An intervertebral disc 120 is located between the second joint surface 106 of the first vertebra 102 and the first joint surface 114 of the second vertebra 112.

Reference to an orientation of the first joint member 100 and the second joint member 110 can be provided by a longitudinal axis 150 and a lateral axis 152 as illustrated.

The cement delivery system flow diagram 400 of FIG. 8 initiates with a step of preparing the surgical site (block 410). This step can include common surgical preparations such as administration of anesthesia, placement of protective barriers, sterilization of the surgical site, creating an incision at the surgical site, and the like. The procedure continues with a step of removing a defective intervertebral disc 120 from between facing vertebrae joint surfaces 106, 114 of the vertebrae 102, 112, respectively, of the joint members 100, 110 (block 412), as illustrated in FIG. 1. The actual direction and method of the removal of the intervertebral disc 120 would be based upon the procedure.

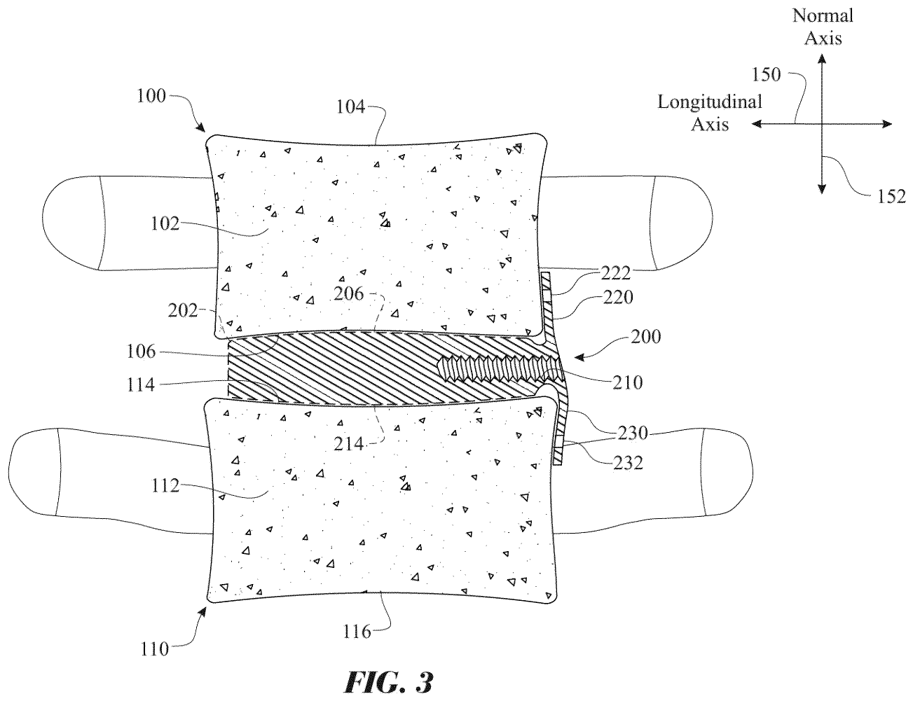

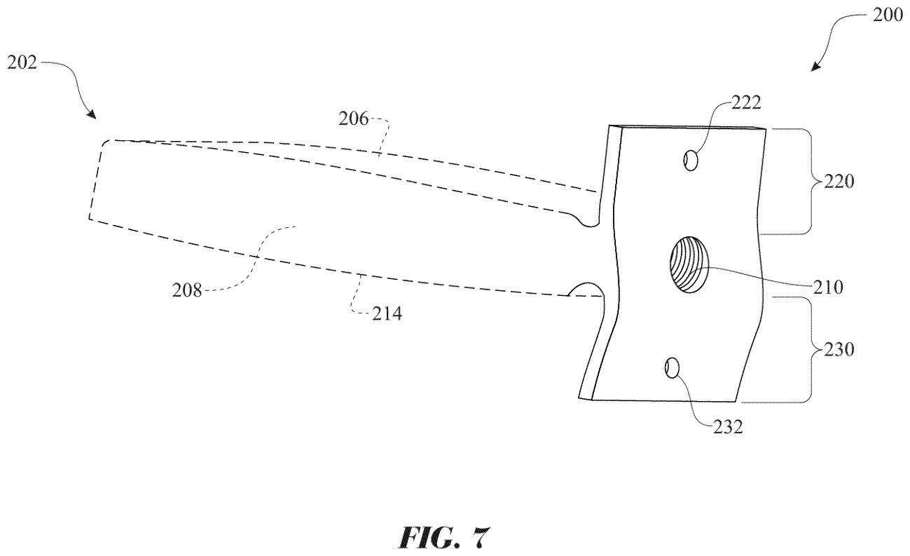

In the exemplary illustrations, a selected intervertebral device body 202 is inserted between the adjacent vertebrae 102, 112 (block 420), as illustrated in FIG. 3. In some procedures, the vertebrae can be distracted (e.g., distracted using one or more dilators) to expand the intervertebral space. The intervertebral device body 202 can be inserted into the expanded intervertebral space such that the first vertebra 102 and the second vertebra 112 captively hold the intervertebral device body 202. The intervertebral device body 202 is a portion of an intervertebral device 200, best illustrated in FIG. 7. The exemplary selected intervertebral device body 202 includes a first disc retention flange 220 extending upward from an edge of the intervertebral device body 202 and a second disc retention flange 230 extending downward from the same edge of the intervertebral device body 202. In some embodiments, the flanges 220, 230 extend along sidewalls of the vertebral bodies of the vertebrae 102, 112. The first disc retention flange 220 and the second disc retention flange 230 can be provided in any of a variety of arrangements and can, for example, include independent elements or be an integrated, singular component comprising both flanges 220, 230. In some embodiments, the flange 220, 230 can form a plate. In an alternate arrangement, in a suitable condition, the intervertebral device 200 can be exclusive of the intervertebral device body 202 and only employ the first disc retention flange 220, the second disc retention flange 230, or an element combining the first disc retention flange 220 and the second disc retention flange 230. In this alternate arrangement, the flange would be placed to retain a defective intervertebral disc 120, such as when the defective intervertebral disc 120 is bulging.

The intervertebral device body 202 is representative of any suitable intervertebral body and can be, for example, a spacer, a cage (e.g., expandable, non-expandable, etc.), an artificial disc, or the like. The intervertebral device body 202 can include an intervertebral device body upper vertebral contacting surface 206, designed to rest against the first vertebra second joint surface 106 of the first vertebra 102, an intervertebral device body lower vertebral contacting surface 214, designed to rest against the second vertebra first joint surface 114 of the second vertebra 112, and a pair of intervertebral device body sidewalls 208 (FIG. 7) extending between the respective elongated edges thereof. The intervertebral device body 202 provides support to each of the first vertebra 102 and the second vertebra 112. The first disc retention flange 220 is placed to abut a side of the first vertebra 102, as illustrated in FIG. 3. The second disc retention flange 230 is placed to abut a side of the second vertebra 112, as illustrated in FIG. 3.

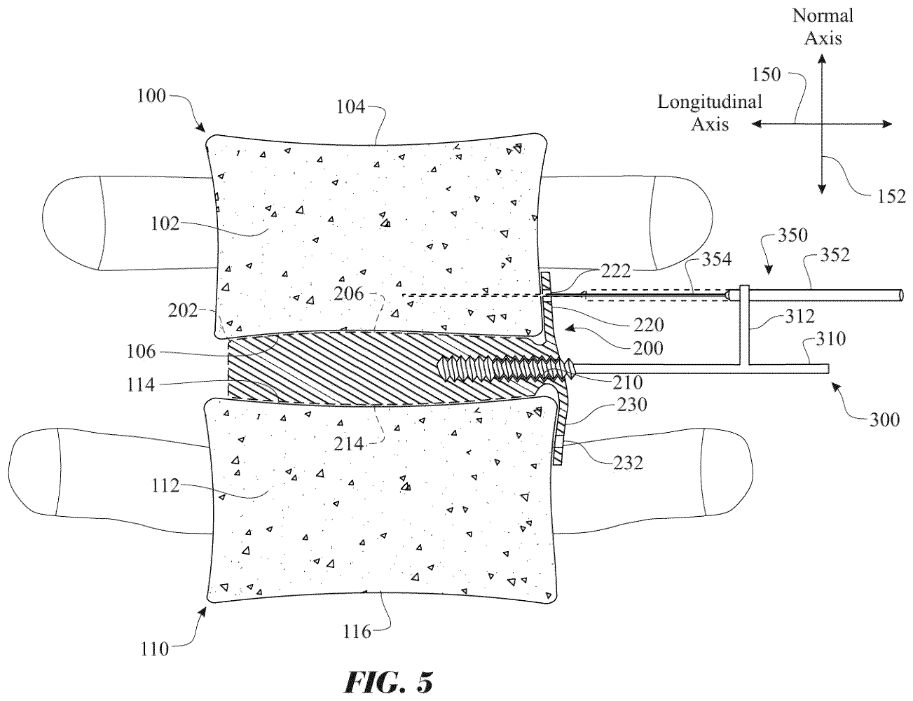

An intervertebral device installation and cement delivery system guide tool 300, introduced in FIG. 5, includes a cement delivery system guide tool component 312 extending generally perpendicularly from an intervertebral device installation component 310. The cement delivery system guide tool component 312 can be located at a longitudinal position on the intervertebral device installation component 310 where a cement delivery conduit 352 of a cement delivery system 350 contacts an interior of an aperture formed through the cement delivery system guide tool component 312 of the intervertebral device installation and cement delivery system guide tool 300 prior to a tip of a cement delivery needle 354 of the cement delivery system 350 entering a first disc retention flange cement injection guide aperture 222 (e.g., throughhole) of the first disc retention flange 220. This can enable the cement delivery system guide tool component 312 to act to pre-align the cement delivery system 350 with the first disc retention flange cement injection guide aperture 222 to avoid, minimize, or limit any damage to the tip of the cement delivery needle 354. The position of the cement delivery system guide tool component 312 can also allow rotation (e.g., 180 degrees of rotation, 360 degrees of rotation, etc.) for needle positioning without interference with the vertebrae 102, 112. Alternatively, the position of the cement delivery system guide tool component 312 would also allow other degrees of rotation to avoid interference with portions of the vertebrae 102, 112.

The intervertebral device installation component 310 can include a threaded end for engagement with an interbody installation tool receiving threaded bore 210 of the intervertebral device body 202. Although the exemplary illustrations present a threaded interface, it is understood that any interface can be utilized between the intervertebral device installation component 310 and the intervertebral device 200. The interface can be configured to allow rotation of the intervertebral device installation and cement delivery system guide tool 300.

Once the flange 220, 230 is properly placed, the cement delivery system 350 is aligned with the first disc retention flange cement injection guide aperture 222 formed through the first disc retention flange 220 (block 420), as illustrated in FIG. 5. The cement delivery system 350 is inserted through a guide feature (e.g., an aperture (not identified)) formed through the cement delivery system guide tool component 312 of the intervertebral device installation and cement delivery system guide tool 300, aligning the cement delivery needle 354 with the first disc retention flange cement injection guide aperture 222 (block 422). The cement delivery system 350 is pushed forward through the first disc retention flange cement injection guide aperture 222 (block 424) and driven into the first vertebra 102. The cement delivery needle 354 of the cement delivery system 350 is driven into the first vertebra 102, placing a tip of the cement delivery needle 354 proximate a center of the first vertebra 102, as illustrated in broken lines. In some procedures, the cement delivery needle 354 is positioned in the spongy bone. For example, the cement delivery needle 354 can be positioned in the spongy cancellous bone and spaced apart from cortical bone of the vertebral endplates.

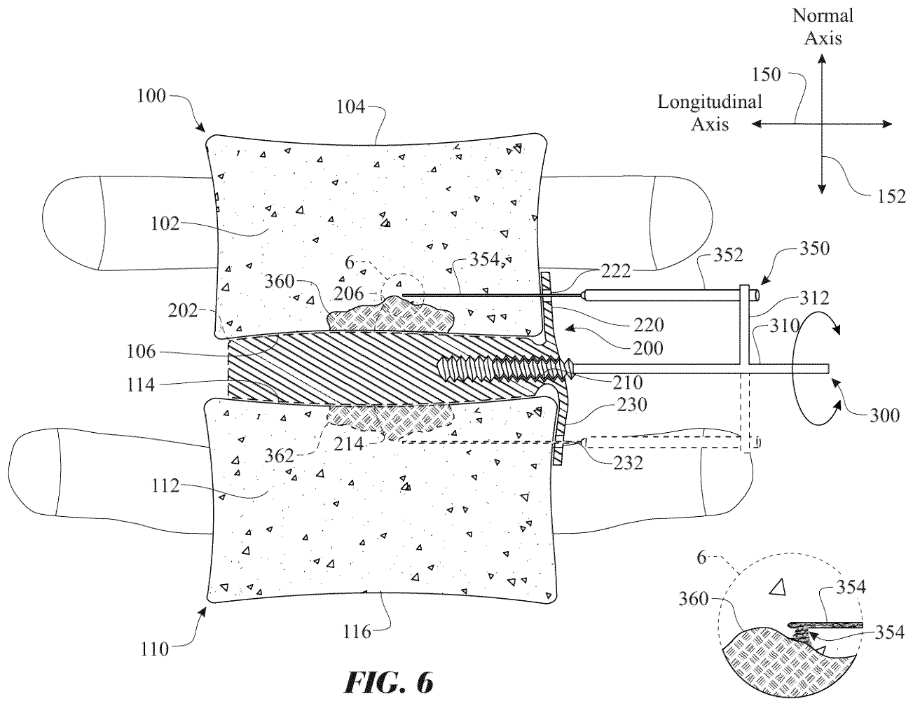

Subsequent to positioning of the tip of the cement delivery needle 354 of the cement delivery system 350, the process dispenses first bone cement deposit 360 into the first vertebra 102 (block 426), as illustrated in FIG. 6. The bone cement can be a flowable material that flows through a region of the spongy cancellous bone. Advantageously, the cortical bone can remain intact to keep the bone cement inside of the vertebral body. In some embodiments, the first bone cement deposit 360 is dispensed into the first vertebra 102 proximate the first vertebra second joint surface 106 to reinforce the material of the first vertebra 102 in the area proximate the first vertebra second joint surface 106, thus increasing support at the interface between the intervertebral device body upper vertebral contacting surface 206 of the intervertebral device body 202 and the first vertebra second joint surface 106 of the first vertebra 102. The cement delivery needle 354 can include a cement delivery needle lumen 356 located on one side of the cement delivery needle 354 proximate the tip of the cement delivery needle 354, as illustrated in a magnified view 6 (in FIG. 6) of the tip of the cement delivery needle 354.

Once the first bone cement deposit 360 is delivered into the first vertebra 102, the cement delivery system 350 is withdrawn from the first vertebra 102, and the process is repeated for the second vertebra 112. The intervertebral device installation and cement delivery system guide tool 300 is rotated to align the aperture through the cement delivery system guide tool component 312 with a second disc retention flange cement injection guide aperture 232 of the second disc retention flange 230 (as indicated by the double-ended arrow in FIG. 6) (block 430).

The cement delivery system 350 is inserted through the aperture (not identified) formed through the cement delivery system guide tool component 312 of the intervertebral device installation and cement delivery system guide tool 300, aligning the cement delivery needle 354 with the second disc retention flange cement injection guide aperture 232 (block 432). The cement delivery system 350 is pushed forward through the second disc retention flange cement injection guide aperture 232 (block 434) and driven into the second vertebra 112. The cement delivery needle 354 of the cement delivery system 350 is driven into the second vertebra 112, placing a tip of the cement delivery needle 354 proximate a center of the first vertebra 102, as illustrated in broken lines in FIG. 6. The process dispenses second bone cement deposit 362 into the second vertebra 112 (block 436) at a location proximate the second vertebra first joint surface 114 to reinforce the material of the second vertebra 112 in the area proximate the second vertebra first joint surface 114, thus increasing support at the interface between the intervertebral device body lower vertebral contacting surface 214 of the intervertebral device body 202 and the second vertebra first joint surface 114 of the second vertebra 112.

Once the second bone cement deposit 362 is delivered into the second vertebra 112, the intervertebral device installation and cement delivery system guide tool 300, including the cement delivery system 350, is withdrawn from the second vertebra 112 (block 440). The surgical site is closed and dressed (block 442).

The result of the cement delivery system flow diagram 400 is a significantly more resolved interface between the opposing joint surfaces 106, 114 of the adjacent vertebrae 102, 112.

The above disclosure describes one exemplary process. Any of a number of modifications can be applied to the concept while maintaining the spirit and intent of the present disclosure. For example, the flanges 220, 230 can be implanted independent of an intervertebral device body 202. In another example, the flanges 220, 230 can be independent of one another, where one or both flanges 220, 230 can be employed. The flanges can be components that are secured to any intervertebral device body 202, such as a fusion spacer, an artificial disc, or any other suitable device.

The bone cement deposits 360, 362 can be applied to one or both vertebrae 102, 112. The bone cement deposits 360, 362 can be any suitable composition, including compositions that differ from bone cement or even compositions that are considered in a cement family. For example, bone graft material, any suitable filler, and the like can be utilized to accomplish the same desired effect. Additionally, the implants can include openings, cavities, and voids for receiving material (e.g., bone graft material, bone cement, etc.). U.S. application Ser. No. 18/670,649; U.S. Provisional Application No. 63/460,330; and U.S. Provisional Application No. 63/528,912 disclose example bone cements, compositions, fillers, and materials that can be used in the procedure. U.S. application Ser. No. 18/670,649; U.S. Provisional Application No. 63/460,330; and U.S. Provisional Application No. 63/528,912 are incorporated by reference in their entireties.

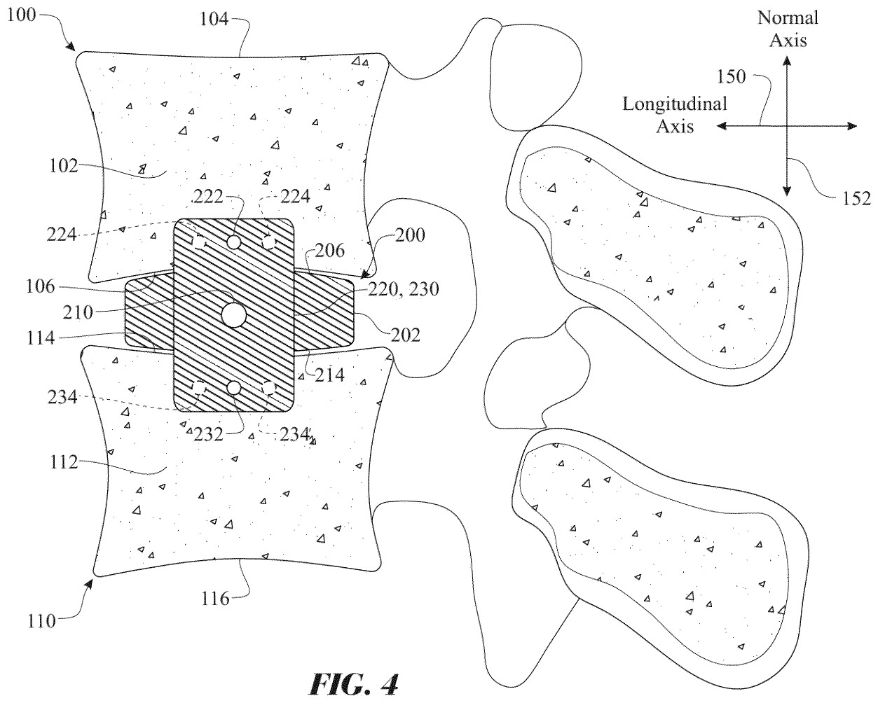

The exemplary illustrations present use of a single aperture 222, 232 for each disc retention flange 220, 230. Additional delivery registration apertures, such as apertures 224, 234 illustrated in broken lines in FIG. 4, can be included in each of the disc retention flanges 220, 230. The apertures 224, 234 can be located along a radius defined by the cement delivery system guide tool component 312, thus utilizing the rotational registration of the intervertebral device installation and cement delivery system guide tool 300 to aid in alignment of the cement delivery needle 354 with the optional outer alignment apertures 224, 234. This also helps follow a contour of the first vertebra second joint surface 106 of the first vertebra 102 and the second vertebra first joint surface 114 of the second vertebra 112, respectively.

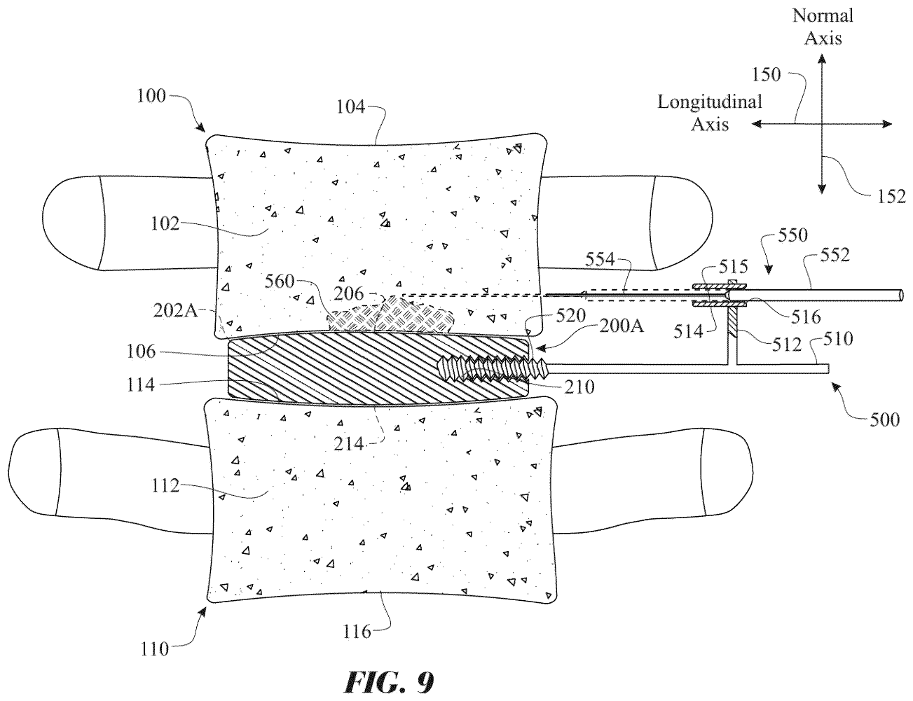

The system can be modified by using an intervertebral device installation and cement delivery system guide tool 500 (similar to the intervertebral device installation and cement delivery system guide tool 300) exclusive of the flanges 220, 230 of the intervertebral device 200, as introduced in FIG. 9. The variation of the intervertebral device 200 introduced in FIG. 9 is an intervertebral device 200A, which eliminates the disc retention flanges 220, 230. The intervertebral device installation and cement delivery system guide tool 500 is rotationally secured to the intervertebral device body 202A of the intervertebral device 200A by threadably assembling an intervertebral device installation and cement delivery system guide tool retention feature 520 (attached to an end of an intervertebral device installation component 510 of the intervertebral device installation and cement delivery system guide tool 500) to the interbody installation tool receiving threaded bore 210. A cement delivery system guide tool component support arm 512 extends radially outward from the intervertebral device installation component 510. A cement delivery system guide tool component 514 is assembled to the cement delivery system guide tool component support arm 512. In the exemplary illustration, the cement delivery system guide tool component 514 is inserted through an aperture formed through the cement delivery system guide tool component support arm 512 and affixed accordingly. A cement delivery system guide tool component bore 515 extends through the cement delivery system guide tool component 514. The cement delivery system guide tool component bore 515 is sized to slidably receive a cement delivery conduit 552 of the intervertebral device installation and cement delivery system guide tool 500. The cement delivery system guide tool component support arm 512 is preferably located at a distance from the intervertebral device installation and cement delivery system guide tool retention feature 520 wherein the cement delivery conduit 552 is supported within the cement delivery system guide tool component bore 515 prior to engagement of a tip of a cement delivery needle 554 of the cement delivery system 550 with the impinging surface of the first vertebra 102.

In use, the cement delivery system 550 can be slidably inserted through the cement delivery system guide tool component bore 515. A cement delivery system guide tool component lead-in 516 can be provided at an entry side of the cement delivery system guide tool component bore 515 to aid in guiding the cement delivery system 550 into the cement delivery system guide tool component bore 515. The intervertebral device installation and cement delivery system guide tool 500 is positioned, aligning the cement delivery needle 554 of the cement delivery system 550 with the first vertebra 102. The tip of the cement delivery needle 554 penetrates the surface (e.g., surface along the sidewall of the vertebral body) of the first vertebra 102 and the cement delivery needle 554 is inserted into the body of the first vertebra 102. The surgeon can determine a depth of the cement delivery needle 554 (or position) based on, for example, a physical marking on the cement delivery conduit 552, a physical marking on the cement delivery conduit 552 in relation with the cement delivery system guide tool component 514, a distance between a mating end provided between the cement delivery conduit 552 and the cement delivery needle 554 and the surface of the first vertebra 102, or any other suitable method (e.g., imaging, fluoroscopy viewing, etc.) of determining an inserted depth and/or position of the cement delivery needle 554. Once the cement delivery needle 554 is positioned to a desired depth, the process dispenses cement into the first vertebra 102 at a location proximate the first vertebra second joint surface 106 forming a bone cement deposit 560. Subsequent to the dispensing process, the cement delivery needle 554 is drawn out from the first vertebra 102. The intervertebral device installation and cement delivery system guide tool 500 is rotated approximately 180 degrees using engagement between the intervertebral device installation and cement delivery system guide tool retention feature 520 and the interbody installation tool receiving threaded bore 210 to rotationally support the intervertebral device installation and cement delivery system guide tool 500, aligning the cement delivery needle 554 of the cement delivery system 550 with the second vertebra 112. The cement injection process is repeated for injection of cement into the second vertebra 112. The cement delivery system 550 is slid rearward to remove the cement delivery needle 554 from the second vertebra 112, and the intervertebral device installation and cement delivery system guide tool 500 is removed from the patient.

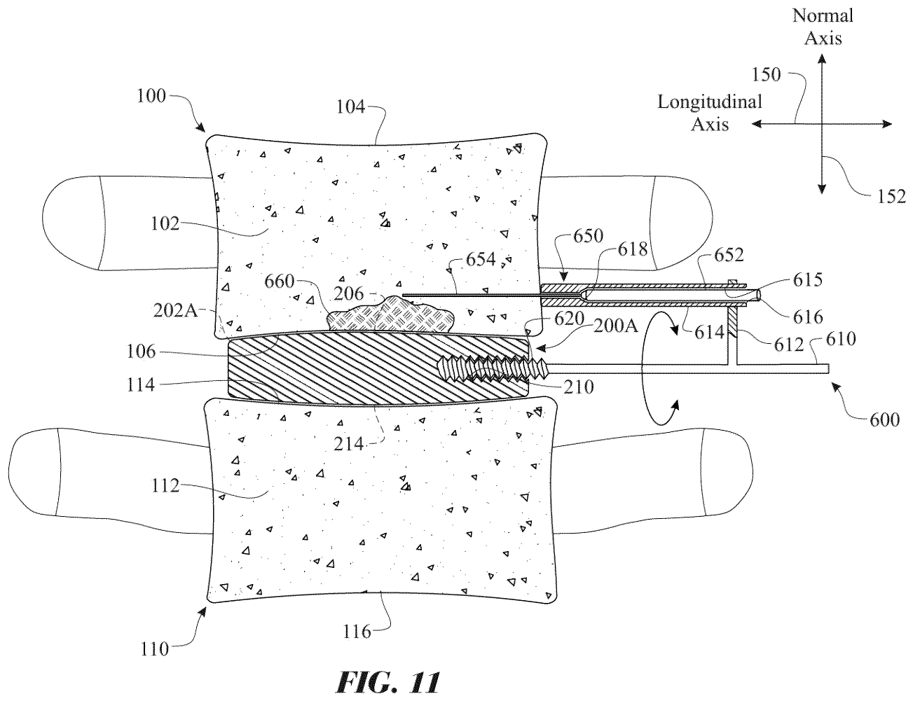

The intervertebral device installation and cement delivery system guide tool 500 can rely upon the surgeon for determining a depth of the cement delivery needle 554 prior to dispensing of the cement. An intervertebral device installation and cement delivery system guide tool 600, introduced in FIG. 11, is similar to the intervertebral device installation and cement delivery system guide tool 500 with minor enhancements. The intervertebral device installation and cement delivery system guide tool 600 and the intervertebral device installation and cement delivery system guide tool 500 include a majority of like elements. Like elements of the intervertebral device installation and cement delivery system guide tool 600 and the intervertebral device installation and cement delivery system guide tool 500 are numbered the same with elements of the intervertebral device installation and cement delivery system guide tool 600 being preceded by the numeral “6.” The cement delivery system guide tool component 614 is extended and includes a cement delivery system guide tool component bore interior stop 618 at a location to limit insertion of a cement delivery needle 654 into the vertebra 102, 112 locating a tip of the cement delivery needle 654 at a desired location within the vertebra 102, 112, as illustrated in FIG. 11. The depth can be established prior to use by adjusting an extended length of the cement delivery needle 654 from the end of the cement delivery conduit 652.

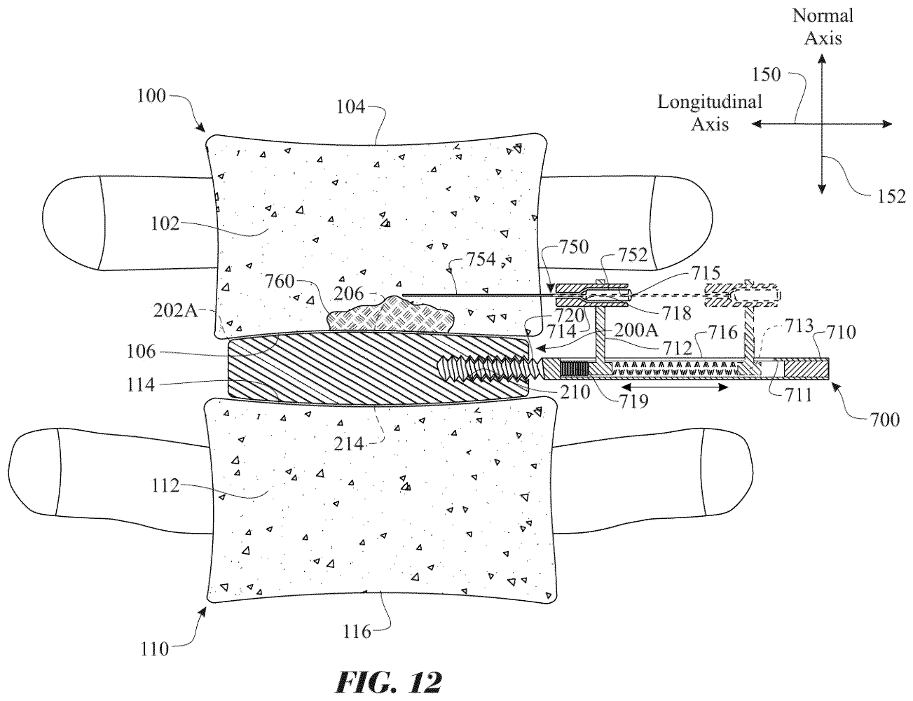

Each of the intervertebral device installation and cement delivery system guide tools 500, 600 can rely upon the surgeon sliding the cement delivery system 550, 650 through the cement delivery system guide tool component 514, 614, respectively. An intervertebral device installation and cement delivery system guide tool 700, introduced in FIG. 12, is similar to the intervertebral device installation and cement delivery system guide tool 600 wherein the intervertebral device installation and cement delivery system guide tool 700 introduces a sliding interface for use to aid in the insertion of a cement delivery needle 754 into the respective vertebra 102, 112. The intervertebral device installation and cement delivery system guide tool 700 and the intervertebral device installation and cement delivery system guide tool 600 include a majority of like elements. Like elements of the intervertebral device installation and cement delivery system guide tool 700 and the intervertebral device installation and cement delivery system guide tool 600 are numbered the same with elements of the intervertebral device installation and cement delivery system guide tool 700 being preceded by the numeral “7.” A cement delivery system guide tool component support arm movement guide element 713 is provided at an end of the cement delivery system guide tool component support arm 712. The cement delivery system guide tool component support arm movement guide element 713 is slidably inserted within an intervertebral device installation component bore interior surface 711 of the intervertebral device installation component 710. A plug (not identified) enables insertion and retains the cement delivery system guide tool component support arm movement guide element 713 within the intervertebral device installation component bore interior surface 711. In some embodiments, a guide tool slide element actuator or biasing member 719 is inserted between facing surfaces of the cement delivery system guide tool component support arm movement guide element 713 and a proximal end of the intervertebral device installation component bore interior surface 711. An intervertebral device installation component guide support arm clearance slot 716 is formed along a portion of a length of the intervertebral device installation component 710, the intervertebral device installation component guide support arm clearance slot 716 providing a passage for the cement delivery system guide tool component support arm 712 therethrough. The cement delivery system guide tool slide element biasing member 719 retains the cement delivery system guide tool component 714 in a distal position until a driving force is applied to the cement delivery system guide tool component 714. Once the cement delivery needle 754 is properly aligned with the respective vertebra 102, 112, the surgeon drives the cement delivery system guide tool component 714 forward against the resistance force created as the cement delivery system guide tool slide element biasing member 719 is compressed, driving the cement delivery needle 754 into the respective vertebra 102, 112. Friction between the cement delivery needle 754 and the respective vertebra 102, 112 can retain the cement delivery needle 754 in position while the cement is deposited to create a bone cement deposit 760. Once the desired volume of cement is dispensed, the cement delivery system guide tool component 714 is retracted, withdrawing the cement delivery needle 754 from the respective vertebra 102, 112. The intervertebral device installation and cement delivery system guide tool 700 can then be rotated 180 degrees and the cement delivery process repeated, delivering the cement into the other respective vertebra 102, 112.

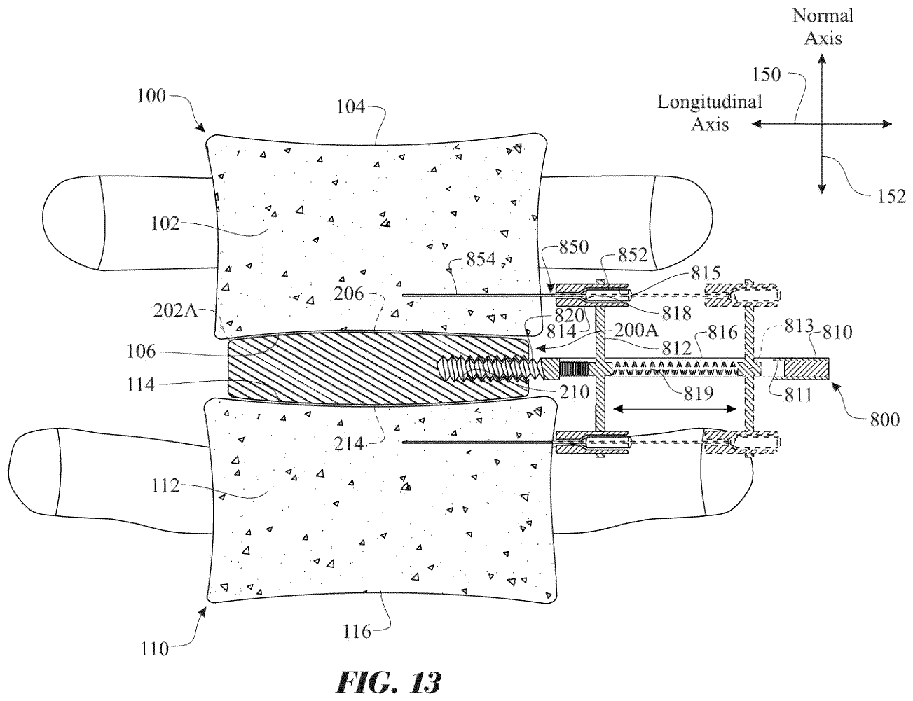

An enhanced version of the intervertebral device installation and cement delivery system guide tool 700 is an intervertebral device installation and cement delivery system guide tool 800, introduced in FIG. 13. The intervertebral device installation and cement delivery system guide tool 800 and the intervertebral device installation and cement delivery system guide tool 700 include a majority of like elements. Like elements of the intervertebral device installation and cement delivery system guide tool 800 and the intervertebral device installation and cement delivery system guide tool 700 are numbered the same with elements of the intervertebral device installation and cement delivery system guide tool 800 being preceded by the numeral “8.” The intervertebral device installation and cement delivery system guide tool 800 replicates the cement delivery system guide tool component 814 and the respective supporting cement delivery system guide tool component support arm 812 at 180 degrees. Each of the opposing cement delivery system guide tool component support arms 812 extends outward from a common cement delivery system guide tool component support arm movement guide element 813. The intervertebral device installation component 810 includes a pair of intervertebral device installation component guide support arm clearance slots 816 to accommodate both cement delivery system guide tool component support arms 812. The intervertebral device installation and cement delivery system guide tool 800 enables the surgeon to inject each of a pair of cement delivery needles 854 into their respective vertebra 102, 112 simultaneously for delivery of the cement. This arrangement avoids a requirement for rotating the intervertebral device installation and cement delivery system guide tool 800 while additionally reducing the time required to complete the procedure.

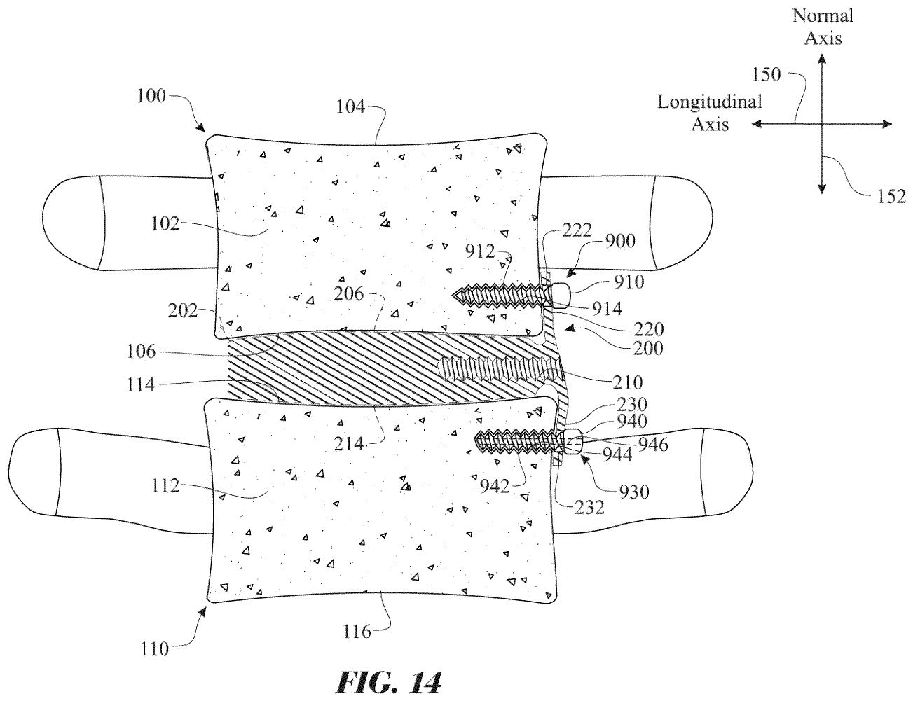

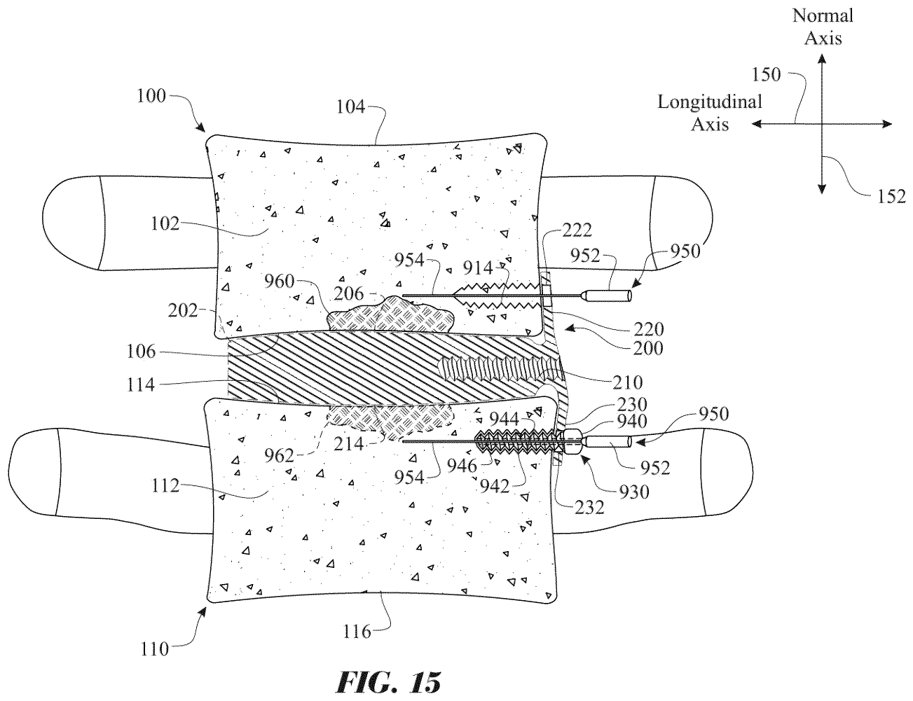

Each of the above procedures utilizes the cement delivery needle 554, 654, 754, 854 for initially piercing the vertebra 102, 112. The present embodiments and technology can be modified by utilizing a secondary element to initiate a passageway for the cement delivery needle 954 of the cement delivery system 950, as introduced in FIGS. 14 and 15. The illustrations in FIGS. 14 and 15 introduce two alternative methods for initiating the passageway for the cement delivery needle 954 of the cement delivery system 950: a threaded attachment member 900 (e.g., a threaded anchor, bone screw, or the like) and a threaded tubular attachment member 930.

Initially describing a process using the threaded attachment member 900, the exemplary threaded attachment member 900 is inserted through the first disc retention flange cement injection guide aperture 222 of the first disc retention flange 220 and threadably inserted by inserting a working end of an insertion tool into a receptacle formed in a threaded attachment member head 910 of the threaded attachment member 900. The insertion tool is used to rotate the threaded attachment member 900, rotating an attachment member threaded body 912 of the threaded attachment member 900, wherein threads of the attachment member threaded body 912 draw the threaded attachment member 900 into the body of the first vertebra 102, creating a threaded cavity 914 within the body of the first vertebra 102. The threaded attachment member 900 is removed, leaving the threaded cavity 914 within the body of the first vertebra 102, as illustrated in FIG. 15. The cement delivery needle 954 of the threaded attachment member 900 is inserted into the threaded cavity 914 and following into the body of the first vertebra 102. Once the cement delivery needle 954 is inserted to the desired depth, cement is dispensed into the body of the first vertebra 102, creating a first bone cement deposit 960. In some procedures, the threaded attachment member 900 (or another threaded attachment member) can be inserted to anchor the flange to the vertebra.

Describing a process using the threaded tubular attachment member 930, the exemplary threaded tubular attachment member 930 is inserted through the second disc retention flange cement injection guide aperture 232 of the second disc retention flange 230 and threadably inserted by inserting a working end of an insertion tool into a receptacle formed in a threaded tubular attachment member head 940 of the threaded tubular attachment member 930. The insertion tool is used to rotate the threaded tubular attachment member 930, rotating a tubular attachment member threaded body 942 of the threaded tubular attachment member 930, wherein threads of the tubular attachment member threaded body 942 draw the threaded tubular attachment member 930 into the body of the second vertebra 112, creating a threaded cavity 944 within the body of the second vertebra 112. A threaded attachment member tubular passage 946 extends axially through the threaded tubular attachment member 930. The cement delivery needle 954, extending in fluid communication from the cement delivery conduit 952 of the cement delivery system 950, is inserted into and through the threaded attachment member tubular passage 946, then into the body of the second vertebra 112 as illustrated in FIG. 15. Once the cement delivery needle 954 is inserted to the desired depth, cement is dispensed into the body of the second vertebra 112, creating a second bone cement deposit 962.

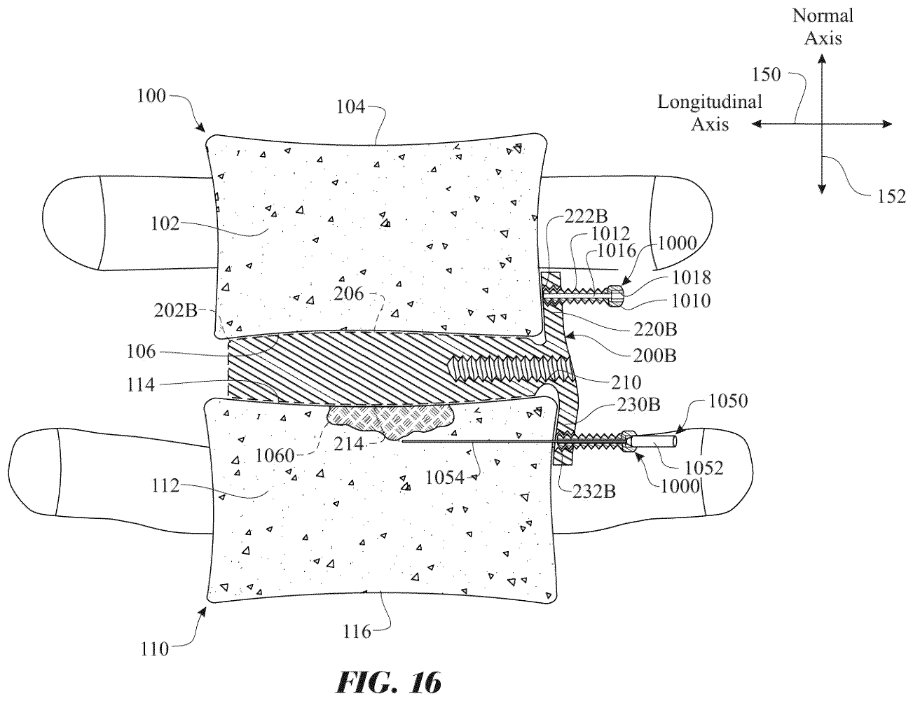

The process can be modified in a form of a hybrid, such as an example presented in FIG. 16. An intervertebral device 200B is a modified version of the intervertebral device 200, the intervertebral device 200B introducing threading through the disc retention flange cement injection guide threaded aperture 222B, 232B of the disc retention flange 220B, 230B, respectively. The disc retention flanges 220B, 230B are preferably attached to or integral with an intervertebral device body 202B of the intervertebral device 200B. Walls of the disc retention flange cement injection guide threaded aperture 222B, 232B may be thicker compared to the walls of the first disc retention flange 220, 230 to accommodate the threading. A threaded cannula 1000 is threadably assembled to the disc retention flange cement injection guide threaded aperture 222B, 232B, as illustrated.

A cannula threaded body 1012 of the threaded cannula 1000 is threadably assembled to the disc retention flange 220B, 230B by inserting a working end of an insertion tool into a threaded cannula drive tool receptacle 1018 formed in a threaded cannula head 1010 of the threaded cannula 1000. The insertion tool is used to rotate the threaded cannula head 1010, rotating the cannula threaded body 1012 of the threaded cannula 1000, wherein threads of the cannula threaded body 1012 engage with threads of the disc retention flange cement injection guide threaded aperture 222B, 232B, assembling the threaded cannula 1000 to the disc retention flange 220B, 230B, respectively. The threaded cannula 1000 is threaded abutting an end of the cannula threaded body 1012 of the threaded cannula 1000 against a surface of the first vertebra 102, while avoiding dislodging the intervertebral device 200B from the proper position between the first vertebra 102 and the second vertebra 112.

A threaded cannula tubular passage 1016 extends axially through the threaded cannula 1000. The cement delivery needle 1054, extending in fluid communication from the cement delivery conduit 1052 of the cement delivery system 1050, is inserted through the threaded cannula tubular passage 1016 and into the body of the second vertebra 112, as illustrated in FIG. 16. Cement is delivered through the cement delivery needle 1054 into the body of the second vertebra 112, creating the bone cement deposit 1060.

The size and shape of implants, implant bodies, features (e.g., each disc retention flange 220, 230) can vary based upon the desired application, subject's anatomy, physician preferences, surgical path, etc. For example, the implants can be cages (e.g., non-expandable or expandable cages), artificial discs, articulating implants, etc. and can be configured for an anterior lumbar interbody fusion (ALIF), an oblique lumbar interbody fusion (OLIF), a lateral or extreme lateral lumbar interbody fusion (LLIF or XLIF), a transforaminal lumbar interbody fusion (TLIF), a posterior lumbar interbody fusion (PLIF), a cervical fusion procedure, or other procedures. For example, each of the disc retention flanges 220, 230 can either include or exclude threading, independent of the variant of the embodiment. The injected material can include polymethylmethacrylate (PMMA), surgical bone cement, zinc-based polyalkenoate bone cement, aluminum-free zinc-based polyalkenoate bone cement (e.g., zinc-based glass polyalkenoate (GPC) cements), acrylic-based cements, calcium phosphate cements, combinations thereof, or the like.

The embodiments, features, systems, devices, materials, methods, and techniques described herein may, in some embodiments, be similar to any one or more of the embodiments, features, systems, devices, materials, methods, and techniques described in the following:
U.S. application Ser. No. 18/670,649, filed May 21, 2024, titled “ORTHOPEDIC SPINAL SURGICAL IMPLANT AND METHOD OF USE”;
U.S. Provisional Application No. 63/460,330, filed Apr. 19, 2023;
U.S. Provisional Application No. 63/528,912, filed Jul. 25, 2023; and
U.S. Application No. 63/565,655, filed Mar. 15, 2024, titled “VERTEBRAL CEMENT INJECTION SYSTEM AND METHOD OF USE.”

All of the above-identified patents and applications are incorporated by reference in their entireties. In addition, the embodiments, features, systems, devices, materials, methods, and techniques described herein may, in certain embodiments, be applied to or used in connection with any one or more of the embodiments, features, systems, devices, or other matter. Although the exemplary application describes an intervertebral application, the concept of employing an aperture through an implant for guidance of a system for injecting a bone reinforcement composition into a region of a bone adjacent to a joint can be implemented for any suitable joint.

The above-described embodiments are merely exemplary illustrations of implementations set forth for a clear understanding of the principles of the technology. Many variations, combinations, modifications, or equivalents may be substituted for elements thereof without departing from the scope of the disclosure. Therefore, it is intended that the invention not be limited to the particular embodiments disclosed as the best mode contemplated for carrying out the invention, but that the invention will include all the embodiments falling within the scope of the appended claims.

What is claimed is:

1. A method of injecting a bone reinforcement composition into a spine of a subject, the method comprising:
positioning an implant device at an intervertebral space between a first vertebra and a second vertebra of the subject;
moving a delivery needle of a bone reinforcement composition delivery system through a guide feature of the implant device and into the first vertebra while the guide feature is positioned outside of the intervertebral space; and
dispensing, using the delivery needle extending into the first vertebra, a volume of a bone reinforcement composition into the first vertebra.

2. The method of claim 1, further comprising:
distracting the first vertebra and the second vertebra to expand the intervertebral space; and
inserting the implant device into the expanded intervertebral space such that the first vertebra and the second vertebra captively hold the implant device while the delivery needle is moved into the first vertebra.

3. The method of claim 1, wherein the guide feature is a throughhole in a flange of the implant device, the method further comprising:
positioning the flange along a sidewall of a first vertebral body of the first vertebra, wherein the delivery needle is moved into the first vertebral body while the throughhole is positioned along the sidewall of the first vertebral body.

4. The method of claim 1, wherein the implant device includes an intervertebral implant and the bone reinforcement composition delivery system includes a guide tool detachably coupled to the intervertebral implant, the method further comprising:
moving the delivery needle into the first vertebra while the delivery needle is held by the guide tool.

5. The method of claim 4, further comprising, after dispensing the volume of the bone reinforcement composition:
separating the guide tool from the intervertebral implant; and
removing the guide tool from the subject.

6. The method of claim 4, further comprising positioning a plate of the implant device alongside the first vertebra, wherein a connector extends between the plate and the intervertebral implant.

7. The method of claim 1, further comprising positioning a plate, which is coupled to the implant device, along the first vertebra such that the guide feature is located alongside the first vertebra.

8. The method of claim 1, wherein the implant device includes an intervertebral implant and a guide tool coupled to the intervertebral implant, wherein the delivery needle is moved along the guide feature of the implant device while the delivery needle is held by the guide tool.

9. The method of claim 8, wherein the guide tool is rotatably coupled to the implant device, the method further comprising:
after dispensing the volume of the bone reinforcement composition, rotating the guide tool relative to the implant device to move the guide tool from a first position to a second position;
moving the delivery needle relative to the guide tool, which is positioned at the second position, into a second vertebra; and
dispensing, using the delivery needle, a second volume of the bone reinforcement composition into the second vertebra.

10. The method of claim 1, further comprising positioning a tip of the delivery needle inside of the first vertebra at a location between superior and inferior vertebral endplates of the first vertebra prior to dispensing the volume of the bone reinforcement composition.

11. The method of claim 1, further comprising delivering a bone anchor through the guide feature of the implant device to anchor the implant device to the first vertebra.

12. The method of claim 1, wherein the implant device includes an intervertebral implant and a guide tool coupled to the intervertebral implant, wherein moving the delivery needle includes
moving the delivery needle through a delivery conduit of the guide tool and into the first vertebra, wherein at least a portion of the volume of the bone reinforcement composition is delivered into the first vertebra while the delivery needle is positioned in the delivery conduit.

13. The method of claim 1, wherein the implant device includes an intervertebral implant and a guide tool coupled to the intervertebral implant, wherein moving the delivery needle includes
moving the delivery needle along a delivery conduit of the bone reinforcement composition delivery system to move a tip of the delivery needle through a portion of the first vertebra, wherein at least a portion of the volume of the bone reinforcement composition is delivered into the first vertebra while the delivery needle is positioned in the delivery conduit.

14. The method of claim 1, wherein the implant device is an intervertebral implant and the guide feature is at least one of:
an aperture in a vertebral flange of the intervertebral implant,
an aperture in a vertebral flange extending outward from the intervertebral implant,
a bore extending into the intervertebral implant,
a tool attached to a bore extending into the intervertebral implant, or
a tool rotationally coupled to a bore extending into the intervertebral implant.

15. A method of injecting a bone reinforcement composition into a vertebra of a subject, the method comprising:
inserting an implant device into the subject to address a defective vertebral disc in an appropriate position respective to the vertebra;
moving a delivery needle of a bone reinforcement composition delivery system through a feature integral with the implant device and into the vertebra; and
dispensing, using the delivery needle in the vertebra, a volume of the bone reinforcement composition into the vertebra at a location proximate an intervertebral surface of the vertebra.

16. The method of claim 15, wherein the implant device includes an intervertebral implant and the bone reinforcement composition delivery system includes a guide tool detachably coupled to the intervertebral implant, the method further comprising:
moving the delivery needle into the vertebra while the delivery needle is held by the guide tool.

17. The method of claim 16, further comprising, after dispensing the volume of the bone reinforcement composition:
separating the guide tool from the intervertebral implant; and
removing the guide tool from the subject.

18. The method of claim 15, further comprising positioning an intervertebral body of the implant device in an intervertebral space adjacent the vertebra such that a plate is positioned alongside the vertebra, wherein a connector extends from the plate to the intervertebral body.

19. The method of claim 15, further comprising positioning a plate coupled to the implant device along the vertebra to position the feature along a sidewall of a vertebral body of the vertebra.

20. The method of claim 15, wherein the implant device is an intervertebral implant and the feature is at least one of:
an aperture in a vertebral flange of the intervertebral implant,
an aperture in a vertebral flange extending outward from the intervertebral implant,
a bore extending into the intervertebral implant,
a tool attached to a bore extending into the intervertebral implant, or
a tool rotationally coupled to a bore extending into the intervertebral implant.

21. A spinal implant system, comprising:
an implant device including
an intervertebral body configured to be implanted at an intervertebral space between a first vertebra and a second vertebra, and
a needle guide coupled to the intervertebral body including a guide feature; and
a vertebral cement injection system couplable to the implant device and including a delivery needle configured to be moved into the first vertebra when positioned in the guide feature and the intervertebral body is at the intervertebral space.

22. The spinal implant system of claim 21, wherein the vertebral cement injection system includes a guide tool detachably coupled to the intervertebral body and configured to hold the delivery needle along an injection trajectory spaced apart from a superior endplate and an inferior endplate of the first vertebra.

23. The spinal implant system of claim 21, wherein the vertebral cement injection system includes an actuator configured to drive the delivery needle through cortical bone of the first vertebra.

24. The spinal implant system of claim 22, wherein the vertebral cement injection system is configured to translate the delivery needle along the injection trajectory.

25. The spinal implant system of claim 21, wherein the needle guide is rotatably coupled to the implant device and configured to rotate between a first position for delivering the delivery needle into the first vertebra and a second position for delivering the delivery needle into the second vertebra.

26. The spinal implant system of claim 21, wherein the needle guide is a plate configured to extend in a superior direction along the first vertebra and an inferior direction along the second vertebra when the intervertebral body is implanted at the intervertebral space.

27. The spinal implant system of claim 21, wherein the intervertebral body is configured to receive bone graft material.

28. The spinal implant system of claim 21, wherein the implant device is an intervertebral implant and the guide feature is at least one of:
an aperture in a vertebral flange of the intervertebral implant,
an aperture in a vertebral flange extending outward from the intervertebral implant,
a bore extending into the intervertebral implant,
a tool attached to a bore extending into the intervertebral implant, or
a tool rotationally coupled to a bore extending into the intervertebral implant.
